(19) United States
(12) Patent Application Publication
(10) Pub. No.: US 2009/0269364 A1
(43) Pub. Date: Oct. 29, 2009

(54) HER-2/NEU MULTI-PEPTIDE VACCINE

(75) Inventors: Christoph Zielinski, Vienna (AT); Otto Scheiner, Perchtholdsdorf (AT); Hubert Pehamberger, Vienna (AT); Heimo Breiteneder, Vienna (AT); Ursula Wiedermann, Vienna (AT)

Correspondence Address: MILBANK, TWEED, HADLEY & MCCLOY LLP, INTERNATIONAL SQUARE BUILDING, 1850 K STRET, N.W., SUITE 1100, WASHINGTON, DC, 20006, US

(73) Assignee: Bio Life Science Forschungs-und Entwicklungsges m.b H

(21) Appl. No.: 12/296,738

(22) PCT Filed: Apr. 11, 2007
(86) PCT No.: PCT/EP2007/003226

§ 371 Date: Apr. 3, 2009

(30) Foreign Application Priority Data
Apr. 13, 2006 (EP) 06007834.2

(51) Int. Cl. A61K 39/00; A61P 35/00

(52) U.S. Cl. 424/185.1

(57) ABSTRACT

The present invention relates to a multi-peptide-multiepitop-vaccine against cancerous diseases associated with HER-2/neu oncogene, i.e. the vaccine comprises a specific combination of peptides presenting different amino acids sequences as occur in the extracellular domain of HER-2/neu protein.

The present invention relates to a multi-peptide vaccine against cancerous diseases associated with the HER-2/neu oncogene.

The tumor antigen HER-2/neu, gene product of erbB2/neu protooncogene, is a 185 kDa protein that belongs to the epidermal growth factor receptor family. It consists of a cysteine rich extracellular domain (ECD) with several glycosylation sites, a hydrophobic transmembrane domain, and an intracellular conserve tyrosine kinase domain. HER-2/neu is weakly detectable in epithelial cells of normal tissues but is overexpressed in 20 to 30% of primary breast, ovarian, colon, lung and prostate cancer, and has been linked with poor prognosis and high risk of cancer relapse. The overexpression seems to be stable and homogenous in primary tumors as well as their metastases. Hence, HER-2/neu is an attractive target of cancer immunotherapy.

Passive immunotherapy with monoclonal antibodies is a routinely performed treatment against cancer diseases. Induction of humoral anti-tumor antibody responses by active peptide immunization has therefore become a favourable treatment concept. At present, therapy with Trastuzumab, also known as Herceptin®, a Her-2/neu specific monoclonal antibody (mAb), is standard for the treatment of advanced breast cancers overexpressing the Her-2/neu oncoprotein. The therapeutic efficacy of this mAb suggests that targeting tumors with antibodies has a significant preventive activity. Nevertheless, the utility of passive immunotherapy is limited by inadequate tissue distribution and the necessity of multiple infusions with associated high costs.

Nowadays there are several vaccine strategies targeting HER-2/neu. Such a vaccine strategy requires the development of a method to overcome immune tolerance to the self-protein HER-2/neu.

EP 01 104 943.4 refers to a vaccine comprising two peptides with a length of 13 and 16 amino acids and a sequence, which occur in the extracellular domain of HER-2/neu protein. The used peptides permit active immunization against cancerous diseases associated with HER-2/neu oncogene, but induce less inhibition of tumor growth than Trastuzumab.

Therefore, object of the present invention is to provide a vaccine against cancerous disease involving an overexpression of HER-2/neu protein, which induces strong immunity to establish a immune memory and thus to avoid the disadvantages of conventional cancer treatments and to offer a convincing alternative to other known vaccination methods. A further object of the present invention is to provide a vaccine, which can be administrated to the patient via oral and nasal (mucosal) routes without loosing its strong immunogenic property.

The present invention is based on the finding that the above object can be achieved if the vaccine is a multi-peptide-multiepitop-vaccine against cancerous diseases associated with HER-2/neu oncogene, i.e. the vaccine comprises a specific combination of peptides presenting different amino acids sequences as occur in the extracellular domain of HER-2/neu protein.

Furthermore, it has been found that in order to improve the immunity response the vaccine should optionally comprises an immune-stimulation adjuvant or delivery system, in case the vaccine should administrated via oral or nasal route without loosing its strong immunogenic property, the vaccine optionally comprises a mucosal adjuvant or a mucosal antigen delivery system which is used as mucosal carrier.

Therefore, the present invention provides a vaccine against cancerous diseases associated with the HER-2/neu oncogene, wherein said vaccine comprises a mixture of at least three different peptides having a length of 9 to 30 amino acids and each sequences occurs in the extracellular domain of HER-2/neu protein, or a variation thereof.

The invented vaccine induces a strong immunity and establishes an immuno-memory against cancerous diseases associated with the HER-2/neu oncogene. Thus, the vaccine provides prophylaxis against these cancerous disease. In addition, the inventive vaccine can be used to treat an already existing cancerous disease or to accompany conventional cancer treatments. Application of the inventive vaccine can completely or partly avoid the above-described considerable disadvantages of conventional/passive cancer immunotherapeutics.

In a preferred embodiment of the invention, at least one of the three peptides has the sequence 378 to 394 of the extracellular domain of HER-2/neu protein or functional variants thereof, another one has the sequence 545 to 560 of the extracellular domain of HER-2/neu protein or functional variants thereof and a further peptide has the sequence 610 to 623 of the extracellular domain of HER-2/neu protein or functional variants thereof.

In particular, the peptide having the sequence from 378 to 394 of the extracellular domain of HER-2/neu protein is ID SEQ 1: PESFDGDPASNTAPLQP.

The peptide having the sequence from 545 to 560 of the extracellular domain of HER-2/neu protein is ID SEQ 2: RVLQGLPREYVNARHC.

The peptide having the sequence from 610 to 623 of the extracellular domain of HER-2/neu protein is ID SEQ 3: YMPIWKFPDEEGAC.

Functional variants of the peptides refer to all substance that produce an immune response based on the same mechanism of action as the mentioned peptides.

Specifically, this refers firstly to all peptides constituting functional variants of the peptide, for example the peptide with sequence numbers ID SEQ 1 to 3 (hereinafter designated as functional peptide variants). These can be obtained by omitting one or more amino acids, inserting additional amino acids, substituting amino acids or other modifications, and produce substantially the same immune response as the peptides themselves, for example the peptides with the sequence number ID SEQ 1 to 3. An example to be mentioned for the production of such variants is the substitution of individual amino acids of the peptides, which is done conservatively, that is, by substituting one amino acid by another having similar properties. The nature of the variant obtained by such a substitution remains substantially unchanged in comparison with the peptides, for example the peptides with the sequence numbers ID SEQ 1 to 3, so that the administration of such a variant obtains substantially the same immune response.

In addition, functional variants refer to DNA or RNA molecules that code for the above mentioned peptides, for example the peptides with sequence numbers ID SEQ 1 to 3, or functional peptide variants thereof (hereinafter designated functional nucleic acid variants). These DNA or RNA molecules may also be present in viral vectors. U.S. Pat. No. 5,869,445 describes a number of such types of variants and the production thereof, the relevant disclosure being incorporated by reference.

In addition, functional variants refer to mimotopes of the above mentioned peptides or the peptide variants thereof (hereinafter designated functional mimotopes variants). These can be for example mimetic proteins (hereinafter designated mimetic protein variants), as are found for example by screening phage-peptide libraries with antibodies formed against the peptides or the peptide variants thereof. The procedure for obtaining such mimotopes is known for example from EP 100 41 342.0, the disclosure of which is incorporated by reference.

In the following the term peptide is used for both—peptide and the functional variant thereof—as described above.

The inventive peptides can also be linked to other peptides or polypeptides or to further chemical groups such as glycosyl groups, lipids, phosphates, acteyl groups or the like, as e.g. polyglycol, polyethylengycol, poly-lactic acid (PLA), poly-lactic-co-glycolic acid (PLGA), lysine dendrimers. These substances do not adversely influence the biologically activity of the peptides.

It is preferred that peptide ID SEQ 1 is additionally linked to any linker known in the art, but preferably to a glycine linker and/or a C-terminal cysteine residue, more preferably to the linker of GGGGGC.

Therefore, the preferred vaccine of the present invention comprises a mixture of peptide ID SEQ 1 linked to a glycine linker and/or a C-terminal cysteine residue, in particular to the linker GGGGGC, and peptide ID SEQ 2 as well as peptide ID SEQ 3.

Moreover, it is preferred that each of the inventive peptides are conjugated to a carrier, particularly for systemic immunization.

The used peptides may be conjugated by every method known in the art, for example by genetic engineering or by chemical means, which includes linking of carrier and functional group by a chemical reaction. By genetic engineering, conjugation of the carrier, which may a protein molecule, to the peptide can be effected by inserting a DNA or RNA sequence coding for the total sequence of the conjugate into the an expression system which then expresses the total conjugate.

Additionally, the peptides may be linked to the carrier via a further linker. Thereby, the linker acts as a spacer that confers flexibility or, if desired, rigidity of the conjugated peptide. The chemical nature of the spacer may vary, depending on the reactivity of the functional groups of the carrier and the peptide, respectively, and depending on the necessity in respect of flexibility or rigidity. As an example, spacing sequences such as C-terminal cysteine residues, or glycine like (G), may be mentioned.

Each of the inventive peptides can be conjugated to the carrier in a single or multiple way in different combinations as mono-, di-, tri or oligomers. Such conjugations are described for example in the publication by Th. H. Turpen, S. J. Reinl, Y. Charoenvit, S. L. Hoffmann, V. Fallarme in Bio/Technology, 1995, Vol. 13, pages 53 to 57, by examples of the conjugation of epitopes to macromolecular carriers, or by Wagner et al, 2005 J. Immunol. 174:976-982. The disclosure of this publication is incorporated herein by reference.

It is preferred that the carrier itself has an immune effect, which means the carrier itself is immunogenic.

The carrier is selected from the group consisting of immunogenic peptides, immune stimulation protein sequences like GPC islands, limpet hemocyanin (KLH), tetanus toxoid (TT), cholera toxin subunit B (CTB), bacteria or bacterial ghosts, liposome, chitosome, virosomes, microspheres, dendritic cells, or their like.

In one preferred embodiment each inventive peptide is preferably conjugated to KLH, TT or CTB as carrier by a chemical reaction.

In another preferred embodiment each inventive peptide is preferably conjugated to a virosome or probiotic lactic acid bacteria (LAB) or bacterial ghosts as carrier.

Virosomes are based on liposomes and contain viral proteins embedded in their membranes. These proteins enable the virosome membranes to fuse with cells of the immune system and thus, deliver their contents—the vaccine-specific antigens, in this case the inventive peptides, directly to their targets. Once the virosomes delivered the antigens, the virosomes are completely degraded within the cells. The origin of the virosomes may be a influenza virus.

Furthermore, it is preferred that the vaccine comprises an adjuvant. Adjuvants are biological substances, which enhance humoral and/or cellular immune responses when given with vaccine antigen, in this case with the inventive peptides. In the present invention it is preferred that the adjuvant is used for a systemic immunization.

The adjuvants are selected from the group consisting of e.g. interleukines, bacterial toxins, bacterial cell walls and particles thereof, lipid particles, aluminium hydroxide, squalen derivate, monophospharyl lipid A, or their like.

Moreover, it is preferred that the vaccine can be administrated oral or nasal routs. However, most of protein and/or peptide antigens including purified vaccine antigens are often poorly immunogenic when administered via oral and nasal routes. Thus, co-administration of mucosal adjuvants are essential to induce effective immune responses.

The advantage of a mucosal vaccine lies in a better compliance of the patient to vaccination and a potentially higher efficacy due to the induction of both systemic and mucosal immune responses, this is also e.g. important for tumors situated at the mucosae.

The mucosal adjuvants might be selected from the group consisting of zonula occuldens toxin, heat labile toxin (LT) produced by enterotoxigenic E. coli, vibrio cholerae cholera toxin (CTB) or non-bacterial origin e.g. liposomes, or their like.

A preferred mucosal adjuvant is cholera toxin B subunit, lactic acid bacteria, bacterial ghost or mutant thereof.

In the present invention it is further preferred that each of the inventive peptide is conjugated to the mucosal adjuvant, i.e. each of the inventive peptide is coupled to the mucosal adjuvant for example by a chemical reaction and not only mixed with the mucosal adjuvant. Hence the mucosal adjuvant acts as carrier for the peptides.

In a further preferred embodiment of the present invention probiotic lactic acid bacteria (LAB) are used as mucosal carriers, in particular as mucosal antigen delivery systems. It is known that some lactic acid bacteria (LAB) possess Th1 promoting properties and have therefore been used for vaccination against the Th-2 biased allergic diseases (Repa et al Vaccine 2002; Daniel et al 2006, Allergy).

In the present invention it is shown that IL-12, promoting Th1 responses, enhance the anti-tumor activity of the multi-peptide vaccine. It is therefore preferred to apply certain lactic acid bacteria, which induce IL-12 induction and Th-1 responses, together with the polypeptide vaccine of the present invention. Of particular interest is to use these bacteria as expression system for production of the peptides and also use them as mucosal delivery system. Thereby an oral/mucosal tumor vaccine can be created.

In particular, in the present invention strains of Lactobacillus plantarum or Lactococcus lactis are preferably used as mucosal carriers.

The production of such mucosal carries and the production of vaccines including such carriers are described e.g. in Repa et al., Vaccine, 2003, 22, pages 87 to 95 or Daniel et al, Allergy, 2006.

Furthermore, it is preferred that an further immunogenic adjuvant is added to the inventive vaccine, more preferably interleukine 12 (IL-12) or an IL-12 agonist or a substance that promotes IL-12 production.

Moreover, the vaccine of the present invention may further comprises additives, which are general used in such application like stabilizer, antidegradant etc.

The inventive vaccine can be produced in diverse ways by genetic engineering or chemical means, e.g. solid-phase synthesis method. Such methods are described for example in U.S. Pat. No. 5,869,445.

An example of a genetically engineered production method is to manipulate microorganismen like E. coli or the above mentioned lactic acid bacteria. These are manipulated so that they express the peptides as such or the total conjugates consisting of peptide and carrier coupled thereto.

The inventive vaccine can also be applied in different ways. The vaccine can be administrated for examples intramuscularly, subcutaneously, orally, intranasally or generally mucosally if the vaccine is in capsule or pill form or dispersed in food like yoghurt, if lactic acid bacteria are used as vaccine carriers, or a specific delivery system is used. If the vaccine contains functional nucleic acid variants of the peptides, it can also be administrated by an ex-vivo procedure, which includes removing cells from an organism, inserting the inventive vaccine into these cells and placing the treated cells back in the organism.

The inventive vaccine with or without co-administration of IL-12 can be administrate to the patient according to any treatment schedule.

However, a repeated treatment with IL-12 in a three week interval alone did not show any beneficial or deleterious effect as it has been reported for a treatment in a one week interval (Boggio et al., J. Exp. Med. 1998; 188: 589-96). Therefore, it is preferred that three inventive peptides are given to the patient 4 times in 14 to 21 days interval and further that a day after the three peptides were given a five-day course of IL-12 co-application or an IL-12 agonist or a formulation that induces IL-12 production follows, wherein at the first two days IL-12 is given in a lower concentration than in the last three days. It is preferred that the first concentration of IL-12 is half as high the second one.

The inventive vaccine can be used for prophylactic or acute treatment of mammals that can develop kinds of cancer associated with the HER-2/neu oncogene or in combination with other chemotherapeutics or for prevention of metastasis following surgical intervention.

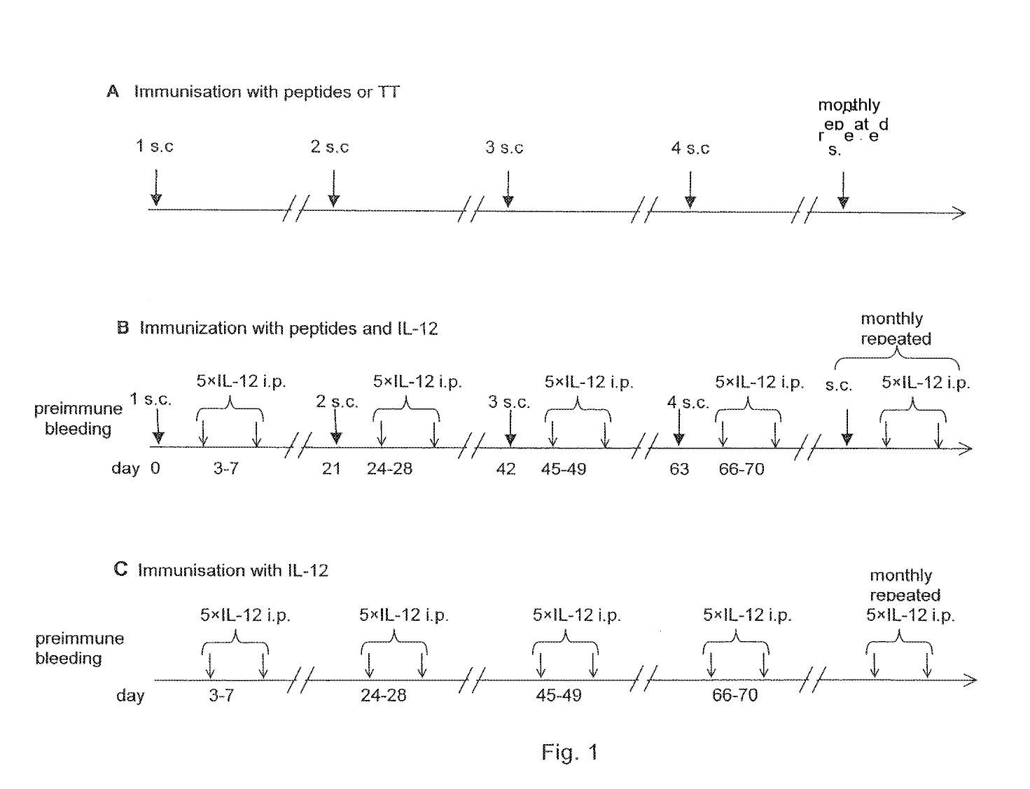

FIG. 1: Experimental design: MMTV-c-neu mice (i.e. Her-2/neu transgenic mice, which spontaneously develop breast cancer) were repeatedly immunized with A) the HER-2/neu peptide conjugates (15 μg each) or TT-alone, B) co-administration with IL-12 or C) with IL-12 alone, in monthly intervals until sacrifice. In separate set-up BALB/c mice were immunized as described for MMTV-c-neu mice and were sacrificed 10 days after the forth immunization.

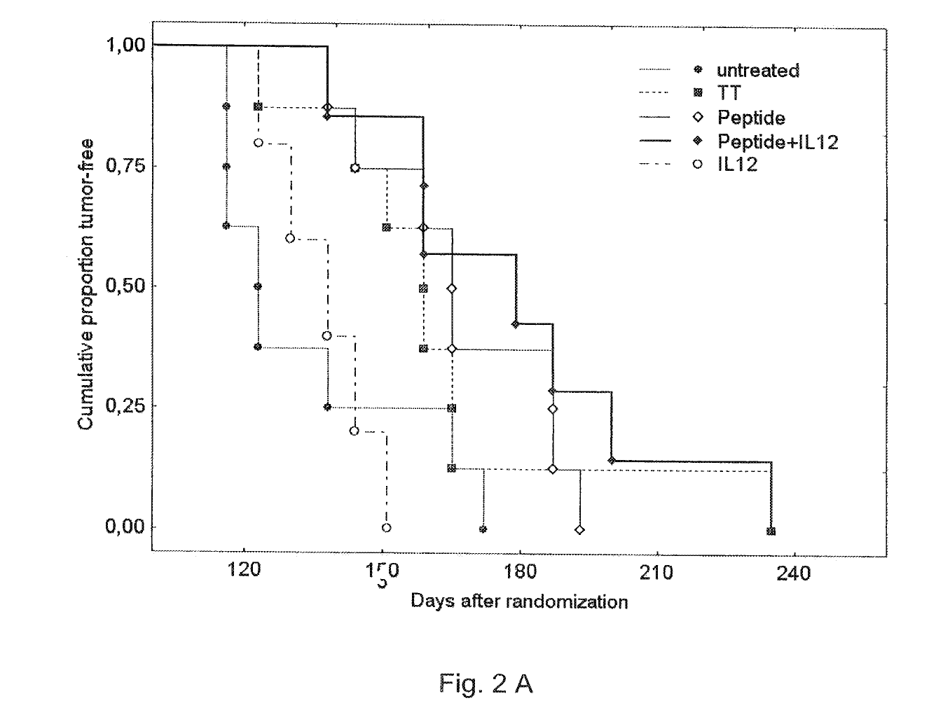

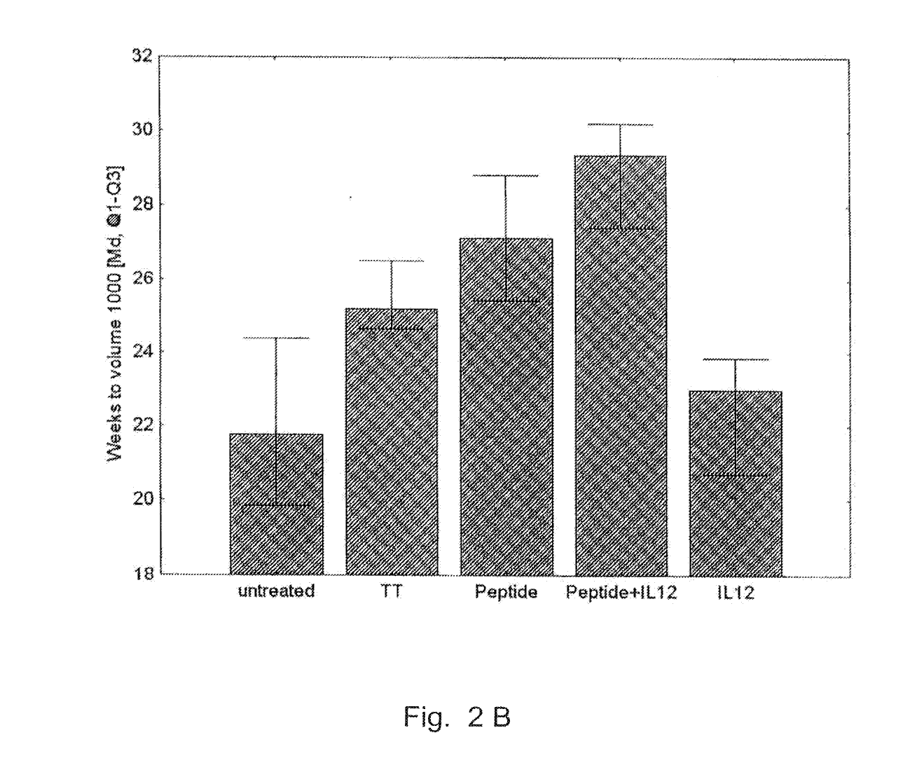

FIG. 2: Immunoprotective effects of HER-2/neu specific vaccination on tumor formation and tumor progression in MMTV-c-neu transgenic mice. Upper panel: The time to tumor development was analysed using Kaplan-Meier survival analysis. Mice (n 8) were vaccinated with TT-conjugated HER-2/neu peptides (⋄), TT-conjugated HER-2/neu peptides and IL-12 co-application (♦), TT alone (▪), IL-12 alone (n=5, o) according to the experimental design given in the FIG. 1. The control group () remained untreated. Lower panel: Tumor progression expressed in weeks until the cumulative tumor volume reached 1000 mm³/mouse. *p<0.05.

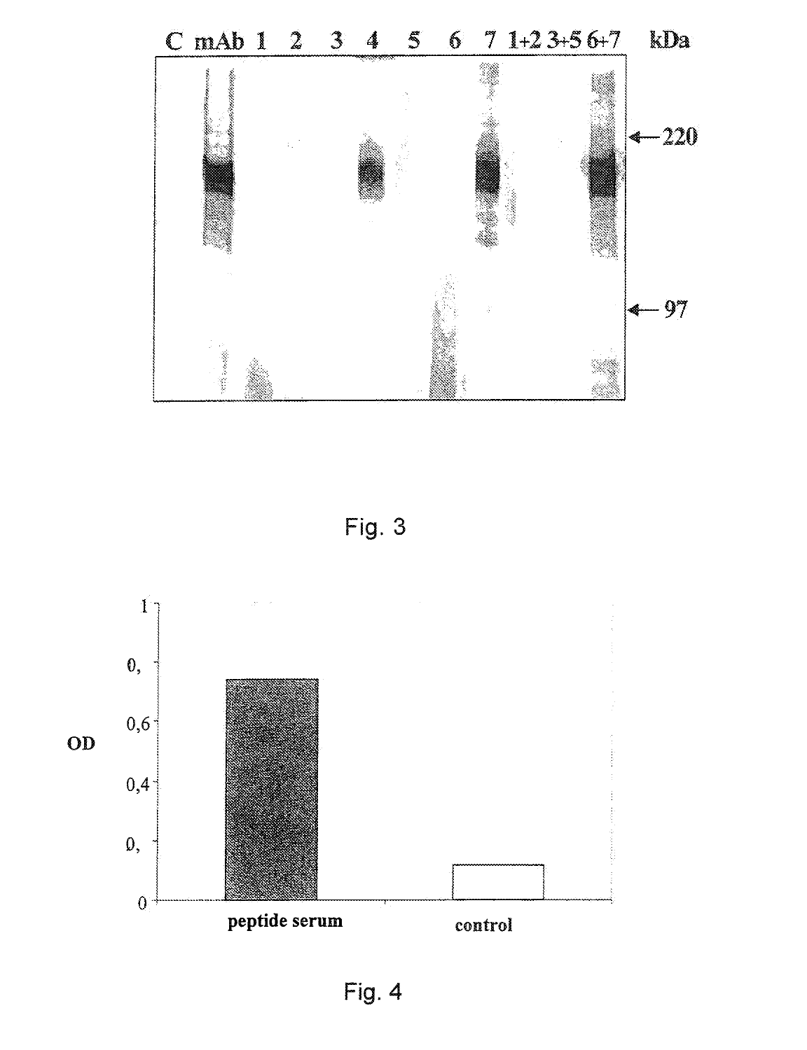

FIG. 3: SK-BR-3 cell lysates were incubated with sera of mice immunized with adjuvant and TT as a control group (lane C), with a murine monoclonal anti human Her-2/neu Ab as a positive control (lane mAb), with sera of mice immunized with conjugated peptides P1−P3, P5 and ID SEQ 1 to 3 (lanes 1-7, lane 4 refers to ID SEQ 1, lane 6 refers to ID SEQ 2 and lane 7 to ID SEQ 3) or a combination of P1+P2 (lane 1+2), P3+P5 (lane 3+5), and ID SEQ2+ID SEQ 3 (lane 6+7). Precipitated Her-2/neu was detected by rabbit anti human Her-2/neu Ab and AP-conjugated swine anti rabbit Ab.

FIG. 4: Her-2/neu specific antibodies measured by ELISA. Peptide serum=sera of mice immunized with peptide/tetanus-conjugate; control serum=sera of mice immunized with tetanus-toxoid.

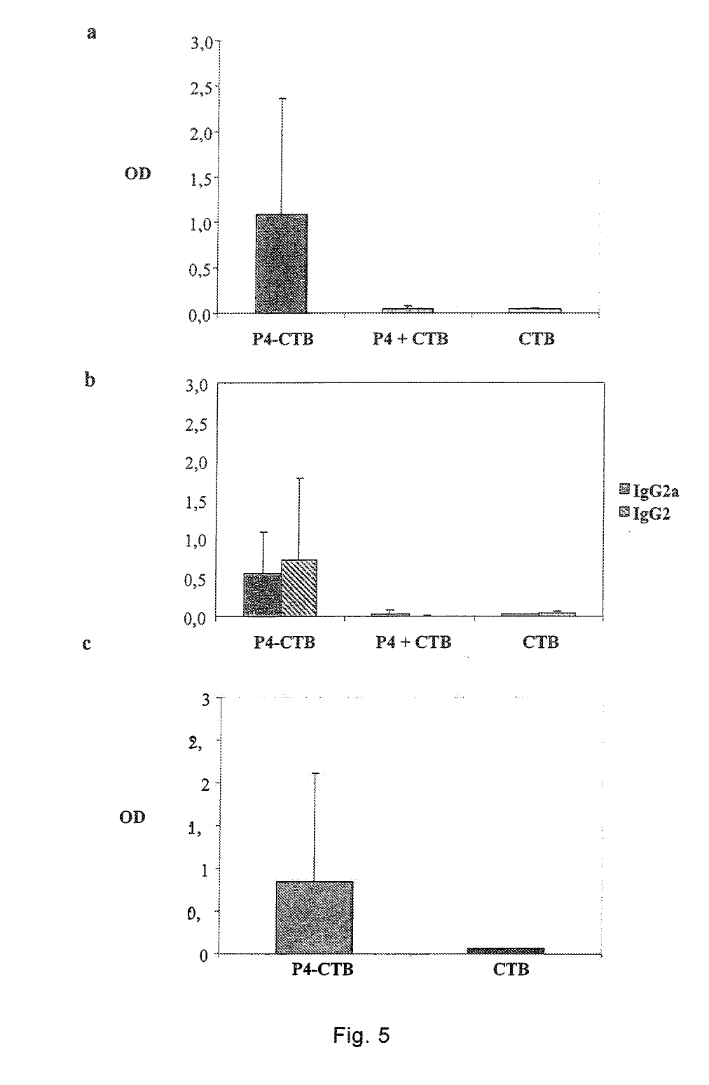

FIG. 5: Immunisation of the mice with the peptide conjugate of ID SEQ 1 and CTB (P4 refers to ID SEQ 1) or a mixture of ID SEQ 1 and CTB (FIG. 5a); the serum dilution was for IgG1 1:10 000, for IgG2a und IgG2b 1:2000 (FIG. 5 b); IgA in bronchoalvelar lavages (1:1) (FIG. 5c)

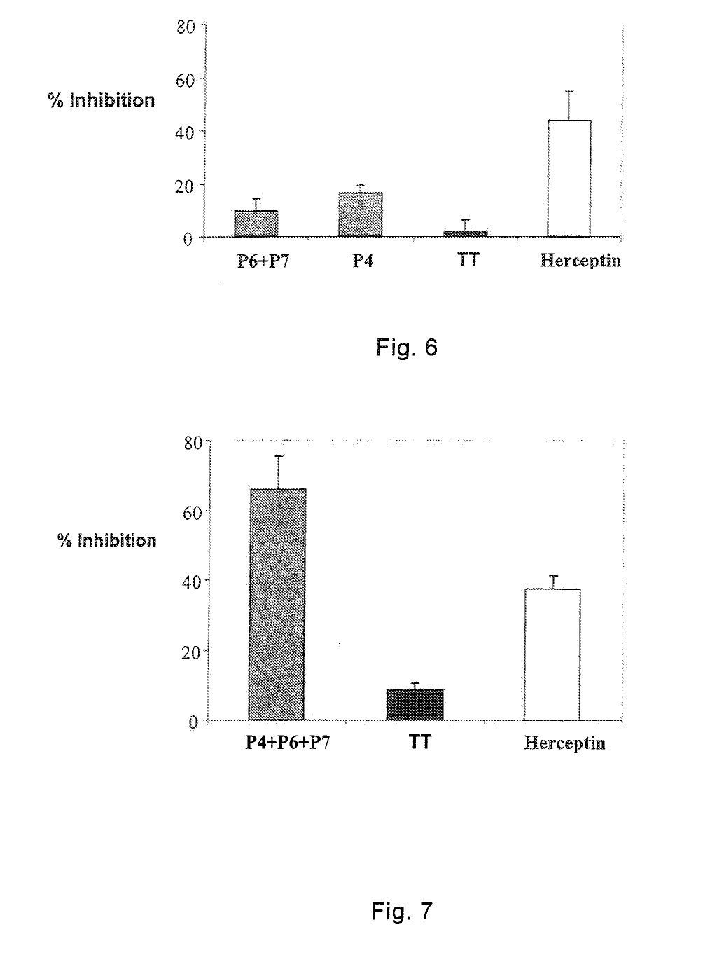

FIG. 6: Inhibition of SK-BR-3 cells with IgG anti ID SEQ 2 and 3 (called P6 and P7) and anti ID SEQ 1 (called P4) and anti TT with a concentration of 150 μg/ml, Trastuzumab (Herceptin) with a concentration of 50 μg/ml.

FIG. 7: Inhibition of SK-BR-3 cells with IgG anti ID SEQ 1 and 2 and 3 (called P4, P6 and P7) and anti TT with a concentration of 75 μg/ml Trastuzumab (Herceptin) with a concentration of 50 μg/ml.

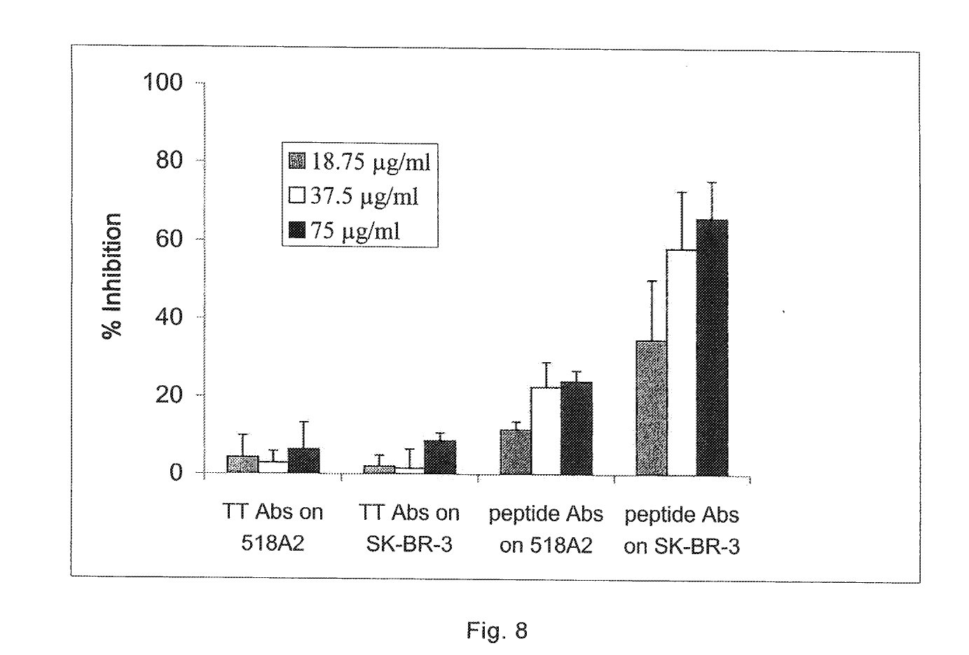

FIG. 8: [3H]-thymidine proliferation assay demonstrating the inhibiting effect in a dose dependent manner of the rabbit IgG on SK-BR-3 cell growth. Data are expressed in percentage of inhibition; cpm values of untreated wells were put to 100%.

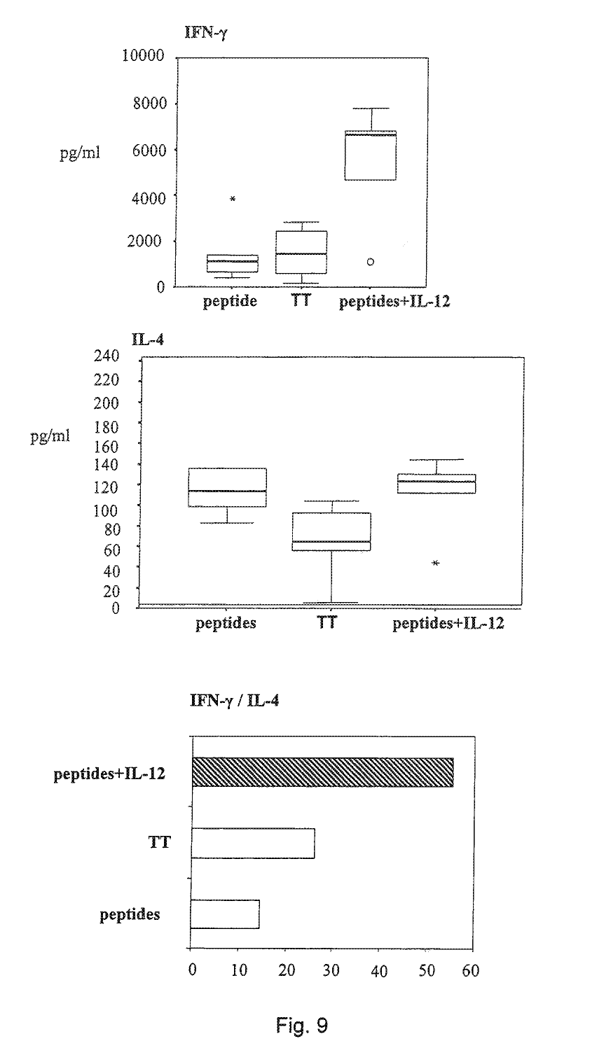

FIG. 9: Cytokine production by spleen cells after in vitro stimulation with TT. BALB/c mice (n=5 per group) were immunized with peptides conjugated to TT with or without systemic co-administration of IL-12 or with TT alone. Splenocytes were cultured with TT and cytokine concentrations in supernatants were evaluated by ELISA. A: IFN-γ levels; B: IL-4 levels; C: IFN-γ/IL-4 ratio. Asterisk indicates p<0.05 (Turkey-Kramer test).

### METHOD AND EXAMPLES

### Mice

The used female FVB/N mice transgenic for the activated rat c-neu oncogene (MMTV-c-neu, 5-9 wk old) and BALB/c mice (8 week old) were purchased from Charles River (Sulzfeld, Germany). Overexpression of the c-neu oncogene was driven by a mouse mammary tumor virus (MMTV) promoter and these mice transgenic for the activated rat c-neu oncogene develop spontaneously mammary tumors by ˜30 weeks of age. Mammary glands were inspected weekly for tumor appearance and progression.

### Tumors

Tumors were measured with a caliper and the volume was calculated by: x²×y/2, whereby x and y represent the short and long dimensions of the tumor. Total tumor volume per mouse was calculated by adding all tumor volumes. Progressively growing masses of >3 mm×3 mm were regarded as tumors. Mice were sacrificed for ethical reasons at the time when a total tumor volume of approximately 2000 mm3 was exceeded. Tumors were excised and stored at −80° C.

All experiments were authorized by the Animal Experimentation Committee of the Medical University of Vienna and the Austrian Ministry of Education, Science and Culture.

### Antigens

The protein sequence of the extracellular domain (ECD) of human HER-2/neu was scanned by computer-aided prediction to search for immunogenic epitopes based on hydrophilicity, accessibility, flexibility, charge distribution or secondary structure propensities. Seven peptides, 14 to 21 amino acids in length, were then synthesized with an additional C-terminal cysteine residue N-alpha-fluorenylmethyloxycarbonyl (Fmoc) chemistry by PiChem (Austria).

TABLE 1

Tested peptides

| | | |
|---|---|---|
| Peptide | Sequence | Amino acids |

| | | |
|---|---|---|
| P1 | 115-132 | AVLDNGDPLNNTTPVTGA |
| P2 | 149-162 | LKGGVLIQRNPQLC |
| P3 | 274-295 | YNTDTFESMPNPEGRYTFGAS |
| ID SEQ 1 | 378-394 | PESFDGDPASNTAPLQP |
| P5 | 489-504 | PHQALLHTANRPEDE |
| ID SEQ 2 | 545-560 | RVLQGLPREYVNARHC |
| ID SEQ 3 | 610-623 | YMPIWKFPDEEGAC |

Furthermore, for the experiments the peptide ID SEQ 1 (PESFDGDPASNTAPLQP) was synthesized with an additional glycine linker and a C-terminal cysteine residue, in particular with the GGGGGC linker.

### Conjugation of Peptides

Peptides were coupled to the carrier proteins tetanus toxoid (TT) or keyhole limpet hemocyanin (KLH) or the B subunit of cholera toxin (such as included in the cholera vaccine Dukoral®) using the heterobifunctional cross-linker reagent m-Maleimidobenzoyl-N-hydroxysuccinimide (MBS) [Pierce, Rockford, Ill.]. The carrier in the mucosal vaccine are coupled to the peptides according to Wagner S et al 2005; J. Immunol. 174:976-982. The amino groups of the carrier proteins were first activated by addition of a 25-fold molar excess of MBS for 30 min at room temperature. Excess MBS was removed by a desalting column (PD-10 column-Amersham Bioscience, Little Chalfont, UK). In a second step peptides were added in a molar ratio of 40:1 peptide-to-carrier protein. Crosslinking occurred to the cysteine residues on the peptides within 3 hours at room temperature. Unbound peptides were removed by dialysis against PBS.

### Vaccination

1. Balb/c Mice (n=5/group) were immunized with three inventive peptides. These peptides were injected individually (25 μg/mouse) or in combination of three peptides (30 μg/mouse). Prior to injection the peptide-conjugates were mixed with Gerbu Adjuvant (Gerbu Technik, Germany) according to the manufacturer's instruction and administered subcutaneously in volume of 100 μl. The control group received the used Carriers and adjuvants alone. The immunizations were performed 4 times in 21 day intervals. Seven days after the last immunization the animals were sacrificed. Blood samples from mice were taken by tail bleeding prior to immunization and seven days after the last immunization.

2. BALB/c Mice (n=5/group) and MMTV-c-neu transgenic mice (n=8/group) were immunized with the combination of the three inventive peptides coupled to tetanus toxoid (TT-conjugates) using 15 μg of each peptide conjugate. Control groups received TT or IL-12 alone or remained unimmunized. Prior to injection, antigens were mixed with Gerbu Adjuvant (Gerbu Technik, Gaiberg, Germany) according to the manufacturer's instruction and administered subcutaneously in a volume of 100 μl. Recombinant mouse IL-12 (Strathmann Biotec, Hamburg, Germany), reconstituted in PBS containing 0.01% mouse serum albumin (Sigma-Aldrich, St. Louis, Mo.), was administered intraperitoneally in a volume of 100 μl. A total of 50 ng of IL-12/injection was given during the first two five-day-courses, followed by 100 ng of IL-12/injections in the subsequent boosts. Immunizations and IL-12 treatments were performed according to the scheme given in FIG. 1. Blood samples were taken by tail bleeding prior to immunization and at the sacrifice. BALB/c mice were sacrificed ten days after the fourth immunization. MMTV-c-neu mice were repeatedly boosted in one-month intervals until sacrifice.

3. Rabbits were immunized with the multi-peptide vaccine or tetanus toxoid and thereafter blood were taken by puncture of an ear-vene prior to and after immunization. Immunizations with the mixture of the peptides, were performed in rabbits at the laboratories of Charles River (Kissleg, Germany). According to the schedule performed in mice the immunizations were done 4 times in 14-21 day intervals. The blood samples were used for in vitro assays.

Spleens, hearts, livers, kidneys and lungs from mice were removed for histopathological analyses.

### Vaccination and Tumor Development

The ability of the inventive vaccine and the effect of co-application of IL-12 to delay spontaneous tumor development and tumor progression was investigated in transgenic MMTV-c-neu FVB/N mice expressing an activated form of rat c-neu that results in a rapid tumor development. The immunization schedule was started with 6-10 weeks old mice according to the experimental design given in FIG. 1 with coupled Her-2/neu peptides combined with or without systemic IL-12 treatment. Mice were boosted monthly until sacrifice. Mammary glands were monitored weekly for number and size of tumors.

First tumors were observed in untreated and control mice receiving IL-12 alone at the age of 22-26 weeks. Within eight weeks (30-34 weeks of age) all mice in these groups developed tumors whereas a significant delay (p<0.05) in tumor onset was observed in the vaccinated groups (FIG. 2A): in 37.5% (3/8) of peptide-immunized mice and in 57.1% (4/7) of the peptide+IL-12 immunized mice the tumor free interval was prolonged by 56 days. In contrast, in the group receiving the carrier protein TT alone only one mouse remained tumor free during this time period.

Monitoring of tumor volumes revealed rapid tumor progression in non-immunized controls. In contrast, peptide-vaccinated groups were characterized by slower progression of established tumors in early stage. As indicated in FIG. 2B, non-immunized controls reached a total tumor volume of 1000 mm³ within 21.5 weeks following the first immunization. This period was exceeded for peptide-immunized mice up to 6.5 weeks (27 weeks after first immunization; p=0.08). In mice treated with the peptide-vaccine co-administered with IL-12 the time period until tumor volume of 1000 mm³ was significantly (p<0.05) prolonged up to 8.5 weeks (29 weeks after first immunization) compared to control mice. No significant reduction in tumor progression was observed in the group of mice receiving IL-12 or TT alone, reaching 1000 mm³ tumor volumes with 23 or 25 weeks after first immunization, respectively.

### Cell Lines

The human breast cancer cell lines SK-BR-3 (HTB 30) and HTB 132 were purchased form ATCC (Manassas, Va., USA). The human melanoma cell lines 518.A2 were kindly provided from B. Jansen (Department of Dermatology, University of Vienna, Austria). The murine mammary tumor cell line Tg1-1 was kindly provided by T. J. Kipps (Division of Hematology/Oncology, University of California, San Diego School of Medicine, CA). Cells were cultured in medium containing 10% fetal bovine serum (PAA Laboratories, Linz, Austria), 50 units/ml penicillin and 50 μg/ml streptomycin (GIBCO, Life Technologies LTD, Paisley, Scotland, UK) in a humidified atmosphere containing 5% CO₂. SK-BR-3 cells were maintained in McCoy's 5A medium, 518.A2, and Tg1-1 cells in DMEM medium. HTB 132 cells were maintained in Leibovitz's L-15 medium and CO₂ free conditions. All media were purchased from GIBCO, Life Technologies LTD, Paisley, Scotland, UK.

### Preparation of Cell Lysates

The human breast cancer cell line SK-BR-3 was used as a source of Her-2/neu protein. Tris buffered saline (TBS, pH 7.4) containing 1% Triton X-100 and 1× Complete EDTA free protease inhibitor mix (Roche, Mannheim, Germany) was used for cell lysis. Approximately 30×10⁶ SK-BR-3 cells were suspended in one ml of lysis buffer, extensively vortexed, and incubated on ice for 15 min. After disruption, samples were centrifuged 10 min at 800 g at room temperature. Supernatants were removed from cell debris and stored at −80° C. until use. Before use cell lysates were diluted 1:3 with 0.1 M TBS pH 7.4 and filtered through 0.45 μm.

### Immunoprecipitation

Aliquots of the cell lysate containing 3.5 mg protein were incubated with 40 μl pooled mice sera or 1 μg anti-c-erbB-2 monoclonal antibody (Zymed, San Francisco, Calif.) and 20 μl protein A+G agarose (Oncogene, Uniondale, N.Y.) and incubated overnight at 4° C. Immunoprecipitates were pelleted and washed twice in 0.1M TBS and in the same buffer containing 0.5% Nonidet P40. Immunoprecipitated proteins were then separated by 6% SDS-PAGE and analysed by Western blotting.

### Western Blot Analysis

Precipitates were separated on 6% SDS-PAGE gels and blotted onto nitrocellulose membranes (Schleicher & Schuell, Dassel, Germany).

Western blots were performed using 40 mM Na₂HPO₄, 7 mM NaH2PO4, 1% milk powder, 0.05% w/v sodium azide, 0.5% w/v Tween-20, pH 7.5 for blocking, washing and antibody dilutions. For detection of precipitated Her-2/neu membranes were incubated with rabbit anti-human Her-2/neu antibodies (diluted 1:100; Zymed, San Francisco, Calif., USA). Bound rabbit Ig was detected by alkaline phosphatase labeled swine anti-rabbit antibody (diluted 1:500; DAKO A/S, Denmark). The substrate 5-bromo-4-chloro-3-indolyl phosphate/nitro blue tetrazolium was converted in situ into a dense blue compound by immunolocalized alkaline phosphatase.

It could be demonstrated that only the three inventive peptides have the capacity to induce specific anti-her-2/neu immune responses (see FIG. 3)

### Microsomal Preparations

Cells were disrupted in lysis buffer (50 mM Na-phosphate ph 7.4, 2 mM EDTA, 250 mM saccharose, 1× Complete protease inhibitor mix (Roche, Mannheim, Germany) using a dounce tissue grindler. Unbroken nuclei were separated by centrifugation at 1,000×g. Thereafter supernatants were centrifuged at 32,000 g for 1 hr at 4° C. Pellets were solubilized in buffer containing 100 mM Na-phosphate pH 7.4, 500 mM NaCl, 2 mM EDTA and 1% Triton X-100. Protein contents were estimated using DC Assay (Bio-Rad Laboraties, Hercules, Calif.) according to manufacturer's instructions. Samples were stored at 4° C. until use.

### ELISA

### a) Peptide Specific Ab Responses

96 well microtiter plates (Nunc-Immuno Plate, Nalge Nunc International, Denmark) were coated with 5 μg/ml peptides conjugated to KLH or 5 μg/ml KLH in 100 mM carbonate buffer, pH 9.6 overnight at 4° C. Nonspecific binding sites were blocked for 4 hr with PBS containing 3% milk powder. Sera from mice immunized with peptide-TT conjugates were diluted 1:100 in PBS-tw (0.05% v/v Tween 20) containing 0.5% milk powder, added to antigen-coated plates and incubated overnight at 4° C.

Sera from mice immunized with peptide-TT conjugates and IL-12 as additional adjuvant were diluted 1:200-1:4000 for measurement of specific IgG2a and 1:8000-1:100.000 for specific IgG1.

Antibody titers in sera of mice immunized i.n. with peptides-CTB conjugates were measured as described above. IgG1 were detected in sera diluted 1:10.000 and IgG2a at a dilation 1:2000. Rat anti-mouse IgG2a or IgG1 (Pharmingen, San Diego, Calif., USA) diluted 1:500 in PBS-tw containing 0.5% milk powder were added to the plates. Bound rat Ig was detected with mouse anti-rat HRP conjugated antibodies (Jackson Immunolab, West Grove, Pa., USA), diluted 1:2000 in PBS-tw containing 0.5% milk powder. Color development was performed with TMB substrate (R&D Systems, MN, USA), the reaction was stopped with 0.1 M H2SO4, and the optical density was measured at 450 nm (630 nm as reference wavelength).

### b) Her-2/Neu Specific Ab Responses

Coating with 5 μg/ml mAB Trastuzumab and blocking of nonspecific binding sites were performed as described above. 96 well plates were then incubated with 50 μg/well microsomal preparation of SK-BR-3 or HTB 132 cells diluted in PBS containing 0.5% Triton X-100 for 2 hr at RT. After washing and blocking, plates were incubated overnight at 4° C. with mouse antisera diluted 1:250 in PBS. Bound Ig was detected with HRP labeled sheep anti-mouse Ig diluted 1:500 in PBS (Amersham Life Science, Buckinghamshire, England). Colour development was performed as described. OD value for each sample are presented after subtraction of control values.

The inventive vaccine induces a significant antibody response to human HER-2/neu in contrast to the control serum (sera of mice immunized with tetanus-toxoid) (FIG. 4). After immunization with the inventive vaccine and IL-12 a tendency towards increased antibody levels of these isotypes was observed.

Also the mucosal application of the peptide/CTB conjugates led to induction of specific antibody responses. Both IgG1 and IgG2a antibodies were induced. It could be demonstrated that the mucosal delivery system has the capacity to induce Th1-like immune responses. The data show that mucosal adjuvants, such as CTB, can induce systemic Th1-biased immune responses and also local IgA. The effects are only achieved, if the peptides are conjugated to CTB and not only admixed to the mucosal adjuvant (FIG. 5 a to c).

### Cell Proliferation Assay

Tumor cells were seeded in 96-well microtiter plates (Costar, Corning, N.Y.) at an optimal density for linear growth: 11×10⁴ cells/well for SK-BR-3 cells and 5×10³/well for 518A2 cells. Cells were allowed to adhere overnight at 37° C. Total IgG isolated from sera of mice and rabbits immunizated with single (FIG. 6) or mixed (FIG. 7, 8) Her-2/neu peptide conjugates or TT was added at a concentration of 150 μg/ml (FIG. 6) or an increasing concentrations (0, 18.75, 37.5, 75 μg/ml) (FIG. 7,8) and Trastuzumab, a humanized anti-Her-2/neu IgG1 mAb purchased from Roche (Hertfordshire, UK), at a concentration of 50 μg/ml. Cells were incubated for 72 h at 37° C., thereafter pulsed for 16 h at 37° C. with 0.5 μCi [3H]-thymidine/well (Perkin Elmer Life Sciences, Boston, Mass.) and afterwards harvested. Incorporated [3H]-thymidin was measured in a 1205 Betaplate Liquid Scintillation Counter (Wallac Oy, Turku, Finland). Percentage of inhibition of proliferation was calculated by comparing the cpm values of treated cells with those of untreated cells, which were put at 100%.

FIG. 6 shows that antibodies induced after immunization with 2 peptides (ID SEQ 2 and 3) or only with 1 peptide (ID SEQ 1) can induce a 20% reduction of the growth of tumor cells in vitro; in comparison, Trastuzumab, leads to 40% tumor growth inhibition in vitro.

FIG. 7 shows that antibodies derived from immunization with a mixture with all three inventive peptides induce a more than 60% tumor growth inhibition, which is even stronger than that induced by the monoclonal ab Trastuzumab. These data show that the combination of the three peptides is of great advantage for the effectiveness of the peptide vaccine and is superior to immunization with single peptides or combinations of only two peptides.

Furthermore, it could be demonstrated that the proliferation of the SK-BR-3 cells expressing high levels of HER-2/neu was inhibited in a dose dependent manner by the peptide specific antibodies ranging from 39% to 66% at 18.75 μg/ml to 75 μg/ml antibody response, respectively (FIG. 8).

### Flow Cytometry

Tg1-1 cells were stripped from culture flasks and washed in staining buffer consisting of HBSS (GIBCO, Life Technologies LTD) and 5% heat-inactivated FCS. 5×10⁶ cells were incubated with 50 μl of pooled immune sera diluted 1:5 in the staining buffer or with 10 μg of the anti-c-erbB-2 monoclonal antibody (NeoMarkers, Fremont, Calif.) in the same buffer. After incubation, cells were washed with the staining buffer and stained with FITC-conjugated bovine anti-mouse antibodies (Santa Cruz, Calif., USA) diluted 1:100 in the same buffer. All incubations were performed on ice for 45 minutes. After the final washing cells were resuspended in HBSS/0.5% FCS. The fluorescence was measured by a FACScan (Becton Dickinson). The mean channel fluorescence intensity and the percentage of positive cells were determined using the CellQuest™ Pro software.

FACS analysis revealed that sera of mice immunized with the inventive vaccine with or without IL-12 were able to bind the c-neu expressing Tg1-1 cells similarly as a monoclonal control Ab directed both against the human and rat HER-2/neu. Weak staining of the cells was also observed using a pool of sera from TT immunized mice while no binding to Tg1-1 with FITC-conjugated anti-mouse IgG antibody was detected (data not shown).

C-neu specificity of the induced antibodies was further proven by the ability to immunoprecipitate c-neu protein from tumor lysates. In contrast, control sera obtained from naïve mice and mice immunized with TT did not show any reactivity with c-neu (data not shown).

### Measurement of Cytokine Release In Vitro

A separate experiment in BALB/mice was performed to study the in vitro cytokine production: Spleens were removed under sterile conditions and prepared as known in the art. Briefly, spleens were homogenized and splenocytes were stimulated with the inventive peptide-vaccine at a concentration of 25 μg/well or with TT at a concentration of 5 μg/well. Control wells were cultured with medium only. Supernatants were collected after 46 h and kept frozen until analysis. IFN-γ levels were measured by ELISA as (not shown). IL-2 and IL-4 levels were measured with commercial mouse ELISA kits (Endogen, Woburn, Mass., USA).

As significant amounts of IFN-g were measured in spleen cell cultures of mice immunized with peptides+IL-12, it could be demonstrated that IL-12 induces INF-gamma production and plays a central role in the improvement of the multiepitope vaccine (FIG. 9).

### Statistical Analysis

Comparisons of tumor free interval for all groups were done by a generalization of Gehan's Wilcoxon test. Group differences for time to tumor volume of 1000 mm³ were analysed by Kruskal-Wallis tests. Post hoc comparisons for all pairs of groups were performed applying Tukey-Kramer tests. The same procedure was applied for cytokines. A p-value below 0.05 was considered significant.

In summary, it could be demonstrated that the inventive vaccine, i.e. the multiepitop vaccine, is effective in preventing c-neu overexpressing tumors in vivo and that this effect could be increased by co-administration of IL-12. The induction of a strong immunity by the inventive vaccine leads to an establishment of immunological memory, potentially preventing tumor recurrence. By extrapolation of the present results active immunization with such a multiepitope vaccine, a prophylactic efficacy but also a good therapeutic efficacy against minimal disease of rapidly growing, drug-resistant tumors is expected. Furthermore, the present vaccine could also be administrated as mucosal vaccine without loosing its high immunization activity, which is an attractive vaccine form for all tumors located at mucosal surfaces.

1-15. (canceled)

16. A vaccine against cancerous diseases associated with the HER-2/neu oncogene, wherein said vaccine comprises a mixture of at least three different peptides having a length of 9 to 30 amino acids and each sequence occurs in the extracellular domain of HER-2/neu protein or a functional variant thereof, wherein at least one peptide has the sequence set forth in amino acid 378 to amino acid 394 of the extracellular domain of HER-2/neu protein or a functional variant thereof, wherein at least one peptide has the sequence set forth in amino acid 545 to amino acid 560 of the extracellular domain of HER-2/neu protein or functional variants thereof, and wherein at least one peptide has the sequence set forth in amino acid 610 to amino acid 623 of the extracellular domain of HER-2/neu protein or a functional variants thereof.

17. The vaccine against cancerous diseases associated with the HER-2/neu oncogene according to claim 16, wherein at least one peptide is coupled to a glycine linker and, optionally, a C-terminal cysteine residue.

18. The vaccine against cancerous diseases associated with the HER-2/neu oncogene according to claim 16 wherein at least one peptide or functional variant thereof is conjugated to a carrier in a single or multiple way.

19. The vaccine against cancerous diseases associated with the HER-2/neu oncogene according to claim 18 wherein the carrier is immunogenic.

20. The vaccine against cancerous diseases associated with the HER-2/neu oncogene according to claim 19 wherein the carrier is selected from the group consisting of keyhole limpet hemocyanin (KLH), tetanus toxoid (TT), B subunit of cholera toxin (CT, CTB), heat labile toxin (LT) or mutants thereof, B subunit (LTB) of E. coli, bacterial ghosts, liposome, chitosomes, virosomes and dendritic cells.

21. The vaccine against cancerous diseases associated with the HER-2/neu oncogene according to claim 16 wherein the vaccine comprises an adjuvant.

22. The vaccine against cancerous diseases associated with the HER-2/neu oncogene claim 21 wherein the adjuvant is a mucosal adjuvant.

23. The vaccine against cancerous diseases associated with the HER-2/neu oncogene according to claim 22 wherein the mucosal adjuvant is cholera toxin subunit B (CTB) or a mutant thereof, or a probiotic lactic acid bacteria.

24. The vaccine against cancerous diseases associated with the HER-2/neu oncogene according to claim 23 wherein the mucosal adjuvant is used as a carrier.

25. The vaccine against cancerous diseases associated with the HER-2/neu oncogene according to claim 16 wherein the vaccine further comprises IL-12 or an IL-12 agonist or a substance that promotes IL-12 production.

26. A method of treating cancerous diseases associated with the Her 2/neu oncogene which method comprises administering to a mammal in need thereof a vaccine according to claim 16 wherein the administration results in a sustained biological response, and wherein the administration comprises:
a. administration of said vaccine 4 times in 14 to 21 day intervals, and
b. subsequent to each administration in step a, administration/or co-administration of or an IL-12 agonist or a substance that promotes IL-12 production in a five-day-course, wherein the IL-12, IL-12 agonist or substance that promotes IL-12 production is administered at two different concentrations.

27. A method of preventing the recurrence of a cancerous disease associated with the Her-2/neu oncongene comprising administering to a mammal in need thereof a vaccine according to claim 16, wherein the administration results in a sustained biological response, and wherein the administration comprises:
a. administration of said vaccine 4 times in 14 to 21 day intervals, and
b. subsequent administration or coadministration of IL-12 or an IL-12 agonist or a substance that promotes IL-12 production in a five-day-course, wherein the IL-12, IL-12 agonist or substance that promotes IL-12 production is administered at two different concentrations.

28. The vaccine according to claim 16, wherein the sequence set forth in amino acid 378 to amino acid 394 of the extracellular domain of HER-2/neu protein is PESFDGDPASNTAPLQP (SEQ ID NO: 1).

29. The vaccine according to claim 16, the sequence set forth in amino acid 545 to amino acid 560 of the extracellular domain of HER-2/neu protein is RVLQGLPREYVNARHC (SEQ ID NO: 2).

30. The vaccine according to claim 16, wherein the sequence set forth in amino acid 610 to amino acid 623 of the extracellular domain of HER-2/neu protein is YMPIWKFPDEEGAC (SEQ ID NO: 3).

31. The vaccine according to claim 17, wherein the glycine linker has the sequence GGGGGC (SEQ ID NO: 4).

32. The method of claim 26, wherein the concentration of IL-12, IL-12 agonist or substance that promotes IL-12 production administered on the first two days of the five-day course is half as high as the concentration that is administered on the last three days of the five-day course.

33. The method of claim 27, wherein the concentration of IL-12, IL-12 agonist or substance that promotes IL-12 production administered on the first two days of the five-day course is half as high as the concentration that is administered on the last three days of the five-day course.

34. A vaccine against cancerous diseases associated with the HER-2/neu oncogene, wherein said vaccine comprises a mixture of at least three different peptides having the following amino acid sequences:

| | |
|---|---|
| PESFDGDPASNTAPLQP; | (SEQ ID NO: 1) |
| RVLQGLPREYVNARHC; | (SEQ ID NO: 2) |
| and | |
| YMPIWKFPDEEGAC, | (SEQ ID NO: 3) |

wherein at least one peptide is conjugated to a glycine linker and optionally, a C-terminal cysteine residue, and wherein the vaccine further comprises a carrier selected from the group consisting of keyhole limpet hemocyanin (KLH), tetanus toxoid (TT), B subunit of cholera toxin (CT, CTB), heat labile toxin (LT), B subunit (LTB) of E. coli, bacterial ghosts, liposome, chitosomes, virosomes, dendritic cells and lactic acid bacteria.

35. The vaccine of claim 34, wherein the glycine linker has the sequence GGGGGC (SEQ ID NO: 4)

36. A method of preventing the recurrence of a cancerous disease associated with the Her-2/neu oncongene comprising administering to a mammal in need thereof a vaccine according to claim 35, wherein the administration results in a sustained biological response, and wherein the administration comprises:
a. administration of said vaccine 4 times in 14 to 21 day intervals, and
b. subsequent administration or co-administration of IL-12 or an IL-12 agonist or a substance that promotes IL-12 production in a five-day-course, wherein the IL-12, IL-12 agonist or substance that promotes IL-12 production is administered at two different concentrations.

37. The method of claim 36, wherein the concentration of IL-12, IL-12 agonist or substance that promotes IL-12 production administered on the first two days of the five-day course is half as high as the concentration that is administered on the last three days of the five-day course.


SEQUENCE LISTING (8 sequences)

SEQ ID NO: 1 - protein, 17 aa, Artificial Sequence
  Her2/neu peptide
PESFDGDPASNTAPLQP

SEQ ID NO: 2 - protein, 16 aa, Artificial Sequence
  Her2/neu peptide
RVLQGLPREYVNARHC

SEQ ID NO: 3 - protein, 14 aa, Artificial Sequence
  Her2/neu peptide
YMPIWKFPDEEGAC

SEQ ID NO: 4 - protein, 6 aa, Artificial Sequence
  Peptide linker
GGGGGC

SEQ ID NO: 5 - protein, 18 aa, Artificial Sequence
  Her2/neu peptide
AVLDNGDPLNNTTPVTGA

SEQ ID NO: 6 - protein, 14 aa, Artificial Sequence
  Her2/neu peptide
LKGGVLIQRNPQLC

SEQ ID NO: 7 - protein, 21 aa, Artificial Sequence
  Her2/neu peptide
YNTDTFESMPNPEGRYTFGAS

SEQ ID NO: 8 - protein, 15 aa, Artificial Sequence
  Her2/neu peptide
PHQALLHTANRPEDE